(19) United States
(12) Patent Application Publication
(10) Pub. No.: US 2022/0012489 A1
(43) Pub. Date: Jan. 13, 2022

(54) APPARATUS AND METHOD FOR MOTOR IMAGERY CLASSIFICATION USING EEG

(71) Applicant: Korea University Research and Business Foundation, Soul (KR)

(72) Inventors: Dong-Joo KIM, Seoul (KR); Seho LEE, Seoul (KR)

(73) Assignee: Korea University Research and Business Foundation, Seoul (KR)

(21) Appl. No.: 17/368,880

(22) Filed: Jul. 7, 2021

(30) Foreign Application Priority Data
Jul. 10, 2020 (KR) 10-2020-0085203

(51) Int. Cl. G06K 9/00; G06K 9/62; G01R 23/02; G06N 3/04; G06N 3/08

(52) U.S. Cl. CPC G06K 9/00536; G06K 9/00523; G06K 9/6217; G06N 3/08; G06K 9/00355; G01R 23/02; G06N 3/0445; G06K 9/0051

(57) ABSTRACT

The present disclosure relates to an apparatus and method for motor imagery classification using electroencephalography (EEG), and more particularly, to an apparatus and method for motor imagery classification that extracts features in different domains included in EEG signals generated during motor imagery in real time and classifies a user's intentions using the features.

### CROSS-REFERENCE TO RELATED APPLICATIONS

This application claims the benefit under 35 USC § 119(a) of Korean Patent Application Number 10-2020-0085203, filed on Jul. 10, 2020, in the Korean Intellectual Property Office, the entire disclosure of which is incorporated herein by reference for all purposes.

### TECHNICAL FIELD

The present disclosure relates to an apparatus and method for motor imagery classification using electroencephalography (EEG), and more particularly, to an apparatus and method for motor imagery classification that extracts features in different domains included in EEG signals generated during motor imagery in real time and classifies a user's intentions using the features.

### BACKGROUND ART

A brain-computer interface is a new communication technology and is aimed at direct connection between brain and computer by controlling a computing device applied to a terminal or a device using electroencephalography (EEG) signals.

Motor imagery prediction technology is technology that classifies a user's imagined movements by analysis of the EEG produced when the user intuitively imagines the intended movements, and corresponds to a higher level technology than other brain-computer interface technologies.

The EEG signals are vulnerable to contamination by background noise such as eye blinking or head movements other than an analysis target due to the measurement limits, and are known as difficult to increase accuracy since EEG analysis for imagined intention classification is difficult.

By this reason, prediction of the user's intentions using noisy EEG signals to increase the accuracy of motor imagery based brain-computer interface classification is still challenging.

The recently developed deep learning technology has improved the accuracy of motor imagery intention prediction technology, but a deep learning model (for example, a deep reliable neural network, a convolutional neural network, a recurrent convolutional neural network, etc.) extracts and learns only single domain features due to its characteristics.

In contrast, the key feature domain in EEG includes spatial domain, temporal domain and frequency (spectral domain). Accordingly, it is necessary to combine various domain features to improve the performance and design a suitable deep learning model for motor imagery intention analysis through EEG signals.

However, the existing technologies which extract features in a signal domain are incapable of extracting information in the other domains, and thus it is impossible to predict motor imagery intention with high performance.

Additionally, the performance may be improved over the single domain technology by combining two types of domains and extracting features, but is still insufficient to apply to motor imagery intention analysis through EEG signals, and the technical limitation of feature selection makes it impossible to effectively use the combined features extracted in large amounts.

Further, the brain-computer interface technology through EEG signal analysis is aimed at reading EEG signals and operating an external device in real time based on the EEG signals. However, the technical limitation of feature selection and low accuracy of real-time feature classification technology hinders the practical application.

### RELATED LITERATURES

### Patent Literatures

(Patent Literature 1) Korean Patent Publication No. 10-2019-0109670
(Patent Literature 2) Korean Patent Publication No. 10-2020-0010640

### DISCLOSURE

### Technical Problem

The present disclosure is designed to solve the above-described problem, and therefore the present disclosure is directed to providing an apparatus and method for motor imagery classification that extracts features in different domains included in electroencephalography (EEG) signals generated during motor imagery in real time and classifies a user's intentions using the features.

### Technical Solution

To achieve the above-described object, a motor imagery classification apparatus using electroencephalography (EEG) for predicting a user's intention by analyzing EEG signals generated during motor imagery in chronological order according to the present disclosure includes an information storage unit to collect EEG signals measured by an EEG measurement device and store the EEG signals, a feature mapping unit to classify the EEG signals into signals measured for each set unit time, combine features measured at a same unit time among features of the EEG signals arranged in chronological order and arrange the features in a matrix structure, a spatial feature analysis unit to set a matrix including the features as each layer to analyze spatial features for each layer, a temporal feature analysis unit to analyze changes in the spatial features between the layers arranged in chronological order, an intention classification unit to classify motor imagery of the measured EEG signals based on input of values of the spatial features changing for each unit time, and a feature point generation reading unit to determine if the EEG signals are generated by motor imagery by analyzing signal changes in a specific frequency band included in the EEG signals.

In this instance, preferably, the feature mapping unit receives time information in which the EEG signals are generated by the motor imagery from the feature point generation reading unit and includes only the EEG signals generated during the motor imagery in the features arranged in the matrix structure.

Additionally, preferably, the feature point generation reading unit includes a frequency analysis unit to analyze a frequency of the EEG signals measured by the EEG measurement device, an energy analysis unit to analyze measured energy in a band region and β band region of the analyzed frequency, and a motor imagery determination unit to analyze energy changes in the α band region and the β band region, and determine that the EEG signals are generated by the motor imagery when the energy in the α band region decreases, and the energy in the β band region increases.

Additionally, preferably, the feature mapping unit assumes that one attempt of the user to imaging moving is made for n sec, classifies the EEG signals into 2n signal blocks, and compares two successive signal blocks.

Additionally, preferably, the feature mapping unit applies a Common Spatial Pattern (CSP) algorithm to extract feature points of multi-channel motor imagery EEG signals.

Additionally, preferably, the feature mapping unit determines a weight of a CSP filter applied to the CSP algorithm, and the weight of the CSP filter is determined by a frequency of the EEG signals generated during the motor imagery.

Additionally, preferably, the spatial feature analysis unit extracts the spatial features for each layer by applying a Convolutional Neural Network (CNN) model to each layer.

Additionally, preferably, the temporal feature analysis unit extracts the temporal features using the changes in the spatial features between temporally successive layers by applying a Recurrent Neural Network (RNN) model to each layer.

Additionally, preferably, the intention classification unit performs classification by a deep learning artificial neural network.

Additionally, preferably, the intention classification unit classifies changes in imagined movements of only one body part as the motor imagery and removes background noise generated by movements or imagination of other body part.

Meanwhile, a motor imagery classification method according to the present disclosure includes an information storage step of collecting EEG signals measured by an EEG measurement device and storing the EEG signals in an information storage unit, a block mapping step of classifying, by a feature mapping unit, the EEG signals into signals measured for each set unit time, combining features measured at a same unit time among features of the EEG signals arranged in chronological order and arranging the features in a matrix structure, a spatial domain analysis step of setting, by a spatial feature analysis unit, a matrix including the features as each layer to analyze spatial features for each layer, a temporal domain analysis step of analyzing, by a temporal feature analysis unit, changes in the spatial features between the layers arranged in chronological order, and a user's intention classification step of classifying, by an intention classification unit, the motor imagery of the measured EEG signals based on input of values of the spatial features changing for each unit time.

### Advantageous Effects

The present disclosure classifies motor imagery intentions using a model which maps features included in electroencephalography (EEG) signals onto a matrix block, extracts the features in the spatial domain in each layer listed in chronological order, and analyzes changes in each feature over time.

Accordingly, since features in many domains are extracted from EEG signals and a user's intentions are predicted from the features in real time, it is possible to improve the performance of motor imagery intention classification and can be effectively used in real-time EEG analysis based on the combined features extracted in large amounts.

### DESCRIPTION OF DRAWINGS

FIG. 1 is a configuration diagram showing a motor imagery classification apparatus using electroencephalography (EEG) according to the present disclosure.

FIG. 2 is a diagram showing a feature mapping unit of the present disclosure.

FIG. 3 is a diagram showing a recurrent convolutional neural network model of the present disclosure.

FIG. 4 shows a motor imagery classification embodiment of the present disclosure.

FIG. 5 shows a comparison table of classification performance between a neural network model of the present disclosure and other models.

FIG. 6 is a flowchart showing a motor imagery classification method using EEG according to the present disclosure.

### BEST MODE

Hereinafter, an apparatus and method for motor imagery classification using electroencephalography (EEG) according to a preferred embodiment of the present disclosure will be described in detail with reference to the accompanying drawings.

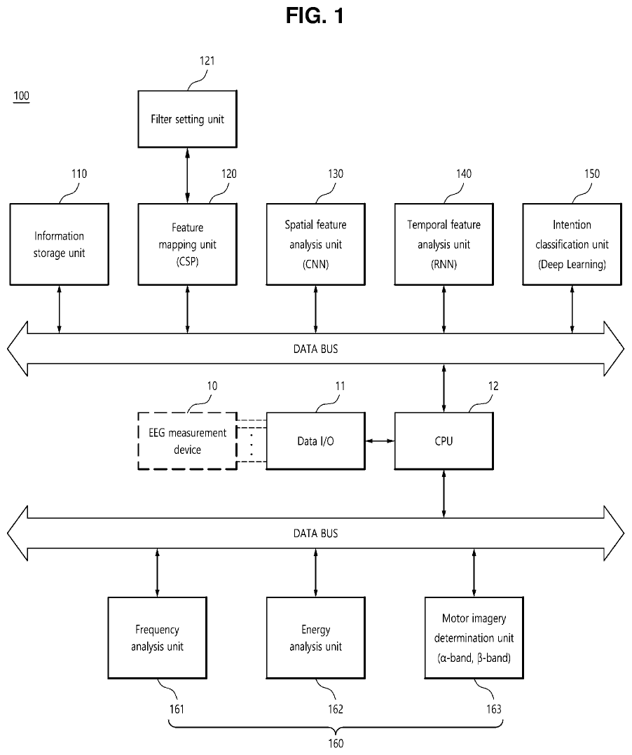

As shown in FIG. 1, the motor imagery classification apparatus 100 using EEG according to the present disclosure includes an information storage unit 110, a feature mapping unit 120, a spatial feature analysis unit 130, a temporal feature analysis unit 140, an intention classification unit 150 and a feature point generation reading unit 160.

The present disclosure predicts a user's intentions by analyzing EEG signals generated during motor imagery in real time, and may be used as an interface to operate assistant devices for paralyzed patients having difficulties in moving the upper limbs as well as external devices including robotic arms, transport means and terminal devices according to the user's intentions.

Additionally, the motor imagery classification apparatus 100 using EEG according to the present disclosure may be implemented in various types, for example, computers capable of receiving and analyzing measured EEG signals, or computing devices of terminals or wearable devices worn or possessed by the user.

Additionally, when the motor imagery classification apparatus 100 using EEG according to the present disclosure is implemented in a computing device, EEG signals measured by an EEG measurement device 10 may be collected through a data I/O 11 and used to control the entire device under the control of CPU. If necessary, a communication module for data communication with the external device may be included.

The present disclosure includes the above-described technical components to map features included in the EEG signals onto a matrix block, extract the features in the spatial domain in each layer listed in chronological order, and analyze changes in each feature over time to classify the user's motor imagery intentions.

Accordingly, the features in many domains may be extracted from the EEG signals and the user's intentions may be analyzed using the features, thereby improving the performance of motor imagery intention classification and effectively using the combined features extracted in large amounts.

To this end, the information storage unit 110 is constructed as a database or a memory to store the user's EEG signals as information to be analyzed. Preferably, the EEG signals are measured by the EEG measurement device, stored in real time and used for analysis.

The EEG is produced when signals are transmitted between brain nerves in the neural system during the brain activity, and is a collection of multi-channel motor imagery signals generated by measuring many parts of the brain using the EEG measurement device at the same time.

The feature mapping unit 120 re-arranges the EEG signals recorded in the information storage unit 110 into a suitable data format for analysis of spatial features and temporal features, and classifies (lists) the features of the EEG signals in the order of unit time and maps the features to the block matrix for each unit time.

That is, the feature mapping unit 120 classifies the EEG signals into EEG signals measured for each set unit time, and among the features of the EEG signals arranged in chronological order, combines the features measured at the same unit time and arranges each feature in a multidimensional matrix structure.

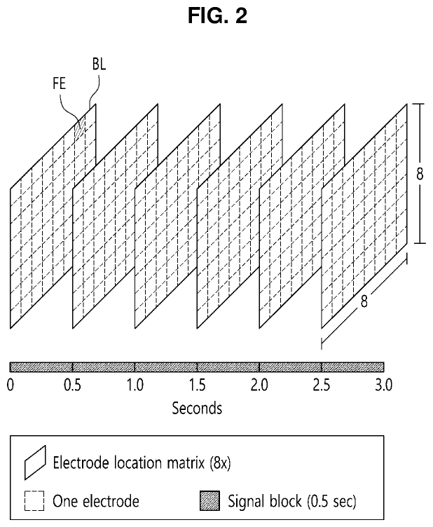

As shown in FIG. 2, since a signal block BL including feature points FE arranged in space has, for example, 8×8 matrix structure, the features are arranged in space, and a plurality of signal blocks are generated for each unit time to analyze feature changes over time.

In this instance, each cell (element) of the matrix that forms the signal block refers to the features FE included in the EEG signals during motor imagery, and for example, is an EEG signal measured by one electrode of the EEG measurement device, and the state of each feature is changed in a particular pattern and/or maintained according to motor imagery.

Additionally, when it is assumed that one attempt of the user to imagine moving is made for n sec, the feature mapping unit 120 classifies the EEG signals into 2n signal blocks and compares two successive signal blocks.

In FIG. 2, 2n signal blocks are generated by the unit time of 0.5 sec, and the features listed in the matrix structure for each signal block are compared to determine motion changes over time.

As a preferred embodiment, the feature mapping unit 120 applies a Common Spatial Pattern (CSP) algorithm to extract the feature points of the EEG signals during multi-channel motor imagery.

The CSP is the most commonly used feature extraction method for motor imagery classification using two classes, and for example, is an algorithm which maximizes the dispersion of one class and minimizes the dispersion of the other class.

Specifically, in the case of EEG which measures brain waves, electrical signals of the brain simultaneously measured at a plurality of electrodes are acquired. In this instance, when it is assumed that T samples are measured at N electrodes, N×T matrix is created at a single measurement, and indicated in matrix. There are a corresponding number of matrices to the number of measurements.

Additionally, when each one task is performed, each covariance C is C=S′/transpose(S′) (here, S=N×T), and covariance of N×T matrix created when X tasks are performed is N×N matrix.

Since there are n (the number of measurements) covariance C and X tasks are performed, an average covariance C1 for the X1-th task and an average covariance C2 for the X2-th task to an average covariance Ci for the Xi-th task are calculated.

The sum of average covariance Csum is transformed into UλU′. Here, U denotes a unique vector, λ denotes a unique value, and a whitening transformation matrix Q is generated using the two unique vectors.

The whitening transformation matrix is calculated through an equation Q=\sqrt{λ⁻¹}U′, and the calculated whitening transformation matrix Q makes each class average covariance matrix have the same unique vector.

Accordingly, when the whitening matrix is applied for different covariance, S=Q*sum of average covariance*Q′(Sᵢ=QC̅ₜQ′), and in this instance, for two classes (maximum dispersion, minimum dispersion), S1=Bλ₁B′, S2=Bλ₂B′.

A weight W of the CSP filter that can be acquired using S1 and S2 is Q′U, and when the acquired W is multiplied by the original N×T signals, the result is CSP filtered Z=WX (here, X=N×T).

Accordingly, n×T (here, n is the number of feature points ‘FE’) CSP filtered signals are acquired, and features are listed in, for example, 8×8 signal block by arranging in a descending order of a difference between the two based on the acquired result. Through this, the EEG features at discrete moments are extracted by extracting the spatially classified feature points.

In this instance, the feature mapping unit 120 of the present disclosure determines the weight of the CSP filter applied to the CSP algorithm according to the frequency of the EEG signals generated during motor imagery.

The filter weight may be separately performed by a filter setting unit 121 connected to the feature mapping unit 120, and the filter setting unit 121 determines the filter weight (or a filter value) by applying a designed function value to the EEG frequency, and reflects the EEG frequency on a model.

The EEG signals have a close relationship with frequencies of 8 to 12 Hz in a band region and 12 to 30 Hz in β band region, and the filter and the frequency are associated to improve the mapping characteristics of the EEG signals generated by motor imagery during feature mapping (CSP algorithm applied). Through this, the present disclosure includes the spatial domain and the temporal domain as well as the spectral domain in motor imagery intention classification to reflect the three domains.

Meanwhile, the spatial feature analysis unit 130 sets the matrix in which the features of the EEG signals are mapped onto the signal block as each layer and analyzes the spatial features for each layer.

Here, spatial feature analysis is used to predict motor imagery at a particular point in time through a distribution or pattern of feature points at each location in the signal block (for example, a 8×8 block) in which the features of the EEG signals are arranged.

For example, when the user imagines moving a hand to generate a command (for example, twist, grip, move, touch, etc.) corresponding to the hand movement, the spatial features may be a shape or image of the hand at a particular point in time.

As described above, the present disclosure may analyze the spatial features for each layer by the spatial feature analysis unit 130, and analyze the temporal features by learning changes between the spatial features in temporally successive layers based on the spatial features.

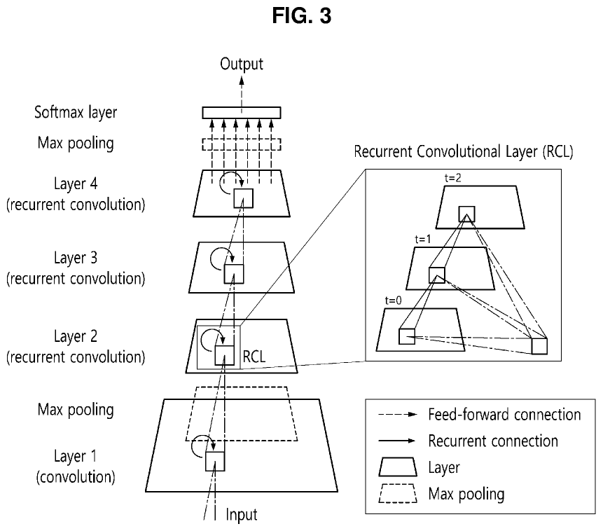

As shown in FIG. 3, the spatial feature analysis unit 130 extracts the spatial features by applying a Convolutional Neural Network (CNN) model to the layers to analyze the spatial features in each layer.

The convolutional neural network forms an output feature map from the features arranged in multi-dimensions using a convolution filter, and forms a new matrix, i.e., an output feature map using matrix multiplication. Additionally, the optimal spatial features for analysis are extracted (analyzed) by adjusting the convolution filter value through learning.

The temporal feature analysis unit 140 analyzes changes in the spatial features between the layers arranged in chronological order, and receives the input of spatial domain feature points in chronological order and extracts temporal feature points.

When the human brain performs a task, brain waves do not disappear immediately after they momentarily change and are maintained for a predetermined time, and the maintenance aspects for the predetermined time are different for each task.

Accordingly, since the spatially made signal block has unique features according to the order of time, the present disclosure extracts the feature points by a temporal domain feature extraction model.

To this end, the present disclosure extracts changes in the features over time by arranging the layers in which the feature points are mapped for each unit time in the order of flow of unit time and comparing the spatial features in each layer.

For example, when the user imagines a command of moving a hand, the spatial features correspond to a shape or image of the hand in each frame, and the temporal features correspond to successive movements of the hand.

To this end, as shown in FIG. 3, the temporal feature analysis unit 140 extracts the temporal features using the changes in the spatial features of the temporally successive layers by applying a Recurrent Neural Network (RNN) model to each layer.

The recurrent neural network is also a class of artificial neural networks where connections between units are recurrent. This structure helps to store the state in the neural network for time-variant dynamic feature modeling.

In particular, the recurrent neural network analyzes a correlation between the temporally successive matrices in time sequence by constructing the neural network in many structures, for example, in a manner of affecting the output of the previous layer on the input of the next layer on the basis of unit time.

For example, when the first image is entered, spatial features are extracted, when the second image is entered, spatial features are extracted in the same way, and temporally correlated features are extracted using the extracted spatial features.

Additionally, when the third image is entered, the model extracts spatial features again, and the current task in the brain, i.e., motor imagery, is predicted by analysis of a correlation with the spatial features extracted from the previous image.

As described above, the present disclosure extracts the spatial features through the convolutional neural network model, and extracts the temporal features using the recurrent neural network model. In this regard, the model of the present disclosure is referred to as a Recurrent Convolutional Neural Network (RCNN) model.

To analyze the user's intention, linear machine learning models using only the features in the temporal or spectral domain have been suggested. However, when movements of body parts are imagined, each region of the brain shows different aspects according to the order of time.

Accordingly, the present disclosure may analyze the EEG produced during motor imagery separately for each of temporal features and spatial features, and predict EEG changes with the user's brain activity by spatial analysis and temporal analysis.

Accordingly, the present disclosure may accurately analyze the user's intention using the temporal/spatial feature extraction method, thereby improving the performance of real-time motion intention analysis, and controlling the external device such as a robotic arm with high accuracy according to the user's intention.

The intention classification unit 150 classifies the motor imagery of the measured EEG signals, and when the recurrent convolutional neural network model is applied, many processing operations such as pooling for reducing the size of the region (for example, max pooling) may be performed for intention classification.

Specifically, the intention classification unit 150 receives the input of the values acquired from the spatial feature analysis unit 130 and the temporal feature analysis unit 140 and classifies the motor imagery intention of the EEG signals using the received values. That is, by the application of the analysis model, motor imagery intention is classified using the input of the values of the spatial features changing for each unit time.

For example, the user's intention is classified by a deep learning artificial neural network using the feature values extracted by the spatial domain feature extraction model and the temporal domain feature extraction model.

Additionally, the intention classification unit 150 classifies changes in imagined movements of only one body part as motor imagery and removes background noise generated by movements or imagination of the other body parts.

One body part refers to a single body part such as any one of the two hands, any one wrist or any one eye, and the present disclosure predicts one body part motor imagery difficult to classify due to higher complexity than distinguishing between many parts.

For example, compared to imaging distinguishing between left and right hands, a different analysis model from the existing analysis model is necessary to analyze EEG produced when imaging the action of twisting the wrist of only the right hand to the right or left.

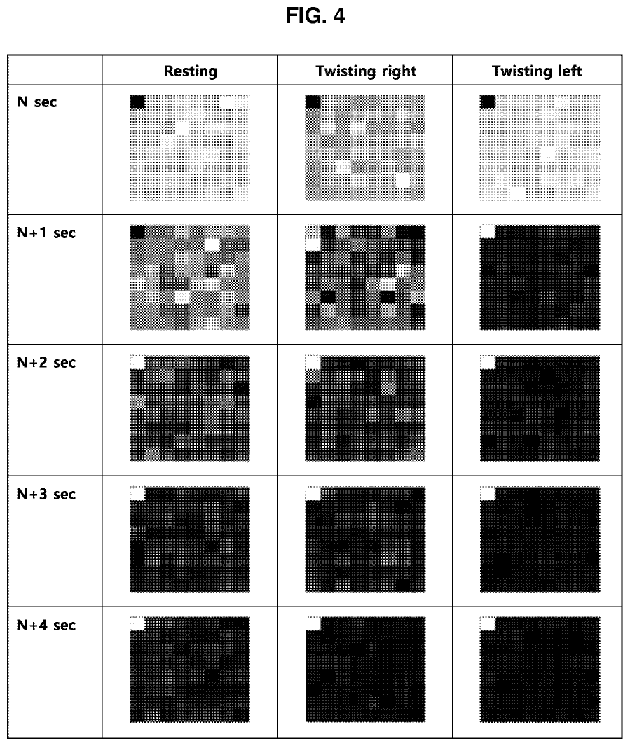

FIG. 4 shows an embodiment in which the user imagines a wrist movement of a hand, and features are mapped to 8×8 signal block through CSP and applied to the RCNN model of the present disclosure.

In an embodiment, the rotation of the wrist is classified into three, resting, twisting right and twisting left, and when it is applied to the model in the order of time from N+1 sec to N+4 sec, the model learns the spatial features and a correlation between a block at the previous time (sec) and the next block.

Accordingly, when the current block is entered as the input, the trained model classifies whether the brain is resting or the hand is twisting to the right or left.

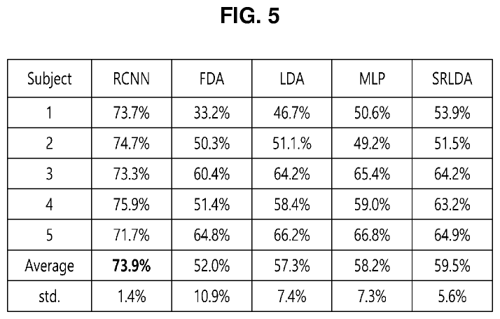

FIG. 5 shows a comparison of classification accuracy result values with other models, and it can be seen that an average value of the RCNN model of the present disclosure is 73.9%, and there is an improvement by about 20% compared to Fisher Discriminant Analysis (FDA), Linear Discriminant Analysis (LDA), Multi-layer Perceptron (MLP) and Shrinkage Regularized Linear Discriminant Analysis (SRLDA).

Meanwhile, the feature point generation reading unit 160 may determine if the EEG signals are generated by motor imagery by analyzing signal changes in a specific frequency band included in the EEG signals, and analyze the EEG signal feature points during motor imagery.

Accordingly, the feature mapping unit may receive time information in which the EEG signals are generated by motor imagery from the feature point generation reading unit 160, and include only the EEG signals generated during motor imagery in the features arranged in a matrix structure based on the time information.

That is, the feature points may be mapped using only the EEG signals generated when the user imagines movements, and the user's motor imagery intention may be classified using the mapped data.

To this end, the feature point generation reading unit 160 may include a frequency analysis unit 161, an energy analysis unit 162 and a motor imagery determination unit 163.

Here, the frequency analysis unit 161 analyzes the frequency of the EEG signals measured by the EEG measurement device. The frequency analysis may be performed by reading data stored in the information storage unit 110 or receiving the input directly from the EEG measurement device.

The energy analysis unit 162 analyzes the measured energy (power or amplitude) in α band region (8 to 12 Hz) and β band region (12 to 30 Hz) of the analyzed frequency. The energy analysis tracks energy increase or decrease in a band region and β band region.

In this instance, α band region is an EEG signal measured all over the head, and μ band region is an EEG signal between 8 and 12 Hz in the central region. Accordingly, since α band region includes μ band region, the present disclosure can track energy increases or decreases in μ band region.

The motor imagery determination unit 163 determines if the EEG signals are generated by motor imagery by analyzing the energy changes in α band region and β band region based on the analysis result of the energy analysis unit 162.

Specifically, during imagination of movements, the motor cortex of the brain have energy decreases in α band region (event related desynchronization) and energy increases in β band region (event related synchronization), and through this, the motor imagery state is determined.

According to the determination result, the computer processing throughput may be optimized by applying the motor imagery intention classification model, or a unit reference time applied to the model may be determined using data at the exact point in time.

In particular, the feature mapping unit receives time information in which the EEG signals are generated by motor imagery from the feature point generation reading unit 160, and includes only the EEG signals while the EEG signals are generated by motor imagery in ‘features’ arranged in a matrix structure.

That is, only the feature points by motor imagery may be extracted and analyzed to prevent noise generated at the other points in time from being included (mixed) in analysis, thereby precisely analyzing the user's motor imagery intention.

Hereinafter, a motor imagery classification method using EEG according to a preferred embodiment of the present disclosure will be described with reference to the accompanying drawings. In this instance, the present disclosure may be performed, for example, in the motor imagery classification apparatus using EEG, and overlapping descriptions are omitted herein.

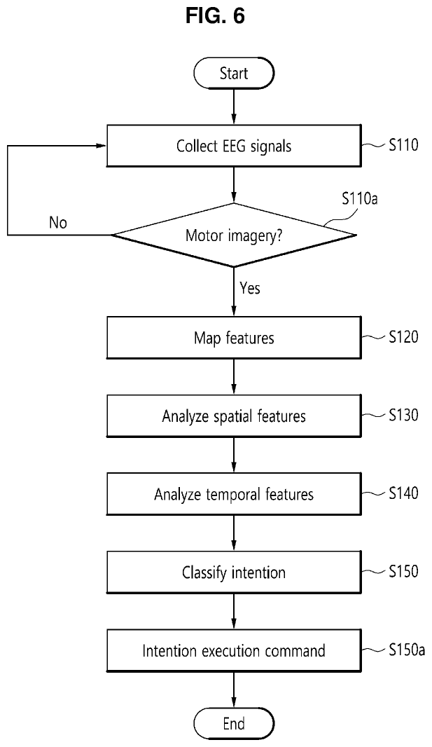

As shown in FIG. 6, the motor imagery classification method using EEG according to the present disclosure includes an information storage step (S110), a block mapping step (S120), a spatial domain analysis step (S130), a temporal domain analysis step (S140) and a user's intention classification step (S150). Preferably, the method further includes a motor imagery determination step (S110A).

As an embodiment, the motor imagery classification method using EEG according to the present disclosure may be implemented in the motor imagery classification apparatus 100 using EEG as described above. Accordingly, it may be implemented in various types of computing devices, including computers, capable of receiving and analyzing EEG signals.

In this instance, in the information storage step (S110), the EEG signals measured by the EEG measurement device are collected and stored in the information storage unit 110. The information storage unit 110 is constructed as a database or a memory to store the user's EEG signals as information to be analyzed.

The EEG is produced when signals are transmitted between brain nerves in the neural system during the brain activity, and is a collection of multi-channel motor imagery signals generated by measuring many parts of the brain by the EEG measurement device at the same time.

Subsequently, in the block mapping step (S120), the EEG signals are classified as signals measured for each set unit time, and among the features of the EEG signals arranged in chronological order, the features measured at the same unit time are combined and each arranged in a matrix structure.

As an embodiment, the feature mapping may be performed by the feature mapping unit 120. The feature mapping unit 120 re-arranges the EEG signals recorded in the information storage unit 110 into a suitable data format for analysis of spatial features and temporal features.

That is, the features of the EEG signals are classified (listed) in the order of set unit time, and the features are mapped to a block matrix for each unit time. Specifically, the EEG signals measured for each unit time are classified, and among the features arranged in chronological order, features measured at the same unit time are combined and each arranged in a multidimensional matrix structure.

In this instance, when it is assumed that one attempt of the user to imagine moving is made for n sec, the feature mapping unit 120 classifies the EEG signals into 2n signal blocks and compares the two successive signal blocks.

As a preferred embodiment, the feature mapping unit 120 applies a Common Spatial Pattern (CSP) algorithm to extract feature points of the EEG signals during multi-channel motor imagery.

The CSP is the most commonly used feature extraction method for motor imagery classification using two classes, and for example, is an algorithm which maximizes the dispersion of one class and minimizes the dispersion of the other class.

Accordingly, the features are listed in the signal block by arranging in a descending order of a difference between the two based on the CSP filtered signal. Through this, the EEG features at discrete moments are extracted by extracting the spatially classified feature points.

In this instance, in the present disclosure, the feature mapping unit 120 determines the weight of the CSP filter applied to the CSP algorithm according to the frequency of the EEG signals generated during motor imagery.

The filter weight may be separately performed in the filter setting unit 121 connected to the feature mapping unit 120, and the filter setting unit 121 may determine the filter weight (or a filter value) by applying a designed function value to the EEG frequency and reflect the EEG frequency on the model.

Subsequently, in the spatial domain analysis step (S130), the spatial features are analyzed by setting the matrix formed by the features of the EEG signals as each layer. As an embodiment, the spatial feature analysis is performed by the spatial feature analysis unit 130.

The spatial domain analysis is used to predict motor imagery at a particular point in time through a distribution or pattern of feature points at each location in the signal block (for example, a 8×8 block) in which the features of the EEG signals are arranged.

For example, when the user imagines moving a hand to generate a command (for example, touch, grip, move, etc.) corresponding to the hand movement, the spatial features may be a shape or image of the hand at the particular point in time.

As described above, the present disclosure analyzes the temporal features by analyzing the spatial features in each layer by the spatial feature analysis unit 130 and learning changes between the spatial features in the temporally successive layers based on the analyzed spatial features.

As shown in FIG. 3, the spatial feature analysis unit 130 extracts the spatial features by applying the Convolutional Neural Network (CNN) model to the layers to analyze the spatial features in each layer.

Subsequently, in the temporal domain analysis step (S140), changes in the spatial features between the layers arranged in chronological order are analyzed. As an embodiment, the temporal domain feature analysis is performed by the temporal feature analysis unit 140.

When the human brain performs a task, brain waves do not disappear immediately after they momentarily change and are maintained for a predetermined time, and the maintenance aspects for the predetermined time are different for each task.

Accordingly, since the spatially made signal block has unique features according to the order of time, the present disclosure extracts the feature points by the temporal domain feature extraction model.

To this end, the present disclosure extracts changes of the features over time by arranging the layers in which the feature points are mapped for each unit time in the order of flow of unit time and comparing the spatial features in each layer.

For example, when the user imagines a command of moving a hand, the spatial features correspond to a shape or image of the hand in each frame, and the temporal features correspond to successive movements of the hand.

To this end, as shown in FIG. 3, the temporal feature analysis unit 140 extracts the temporal features using the changes in the spatial features of the temporally successive layers by applying the Recurrent Neural Network (RNN) model to each layer.

For example, when the first image is entered, spatial features are extracted, when the second image is entered, spatial features are extracted in the same way, and temporally correlated features are extracted using the extracted spatial features.

Additionally, when the third image is entered, the model extracts spatial features again, and the current task in the brain, i.e., motor imagery, is predicted by analysis of a correlation with the spatial features extracted from the previous image.

As described above, the present disclosure extracts the spatial features through the convolutional neural network model and extracts the temporal features using the recurrent neural network model. In this regard, the model of the present disclosure is referred to as a Recurrent Convolutional Neural Network (RCNN) model.

Subsequently, in the user's intention classification step (S150), the motor imagery of the measured EEG signals is classified using the input of the values of the spatial features changing for each unit time. As an embodiment, motor imagery intention classification is performed by the intention classification unit 150.

The intention classification unit 150 classifies the motor imagery of the measured EEG signals, and when the recurrent convolutional neural network model is applied, many processing operations such as pooling for reducing the size of the region (for example, max pooling) may be performed for intention classification.

Specifically, the intention classification unit 150 receives the input of the values acquired from the spatial feature analysis unit 130 and the temporal feature analysis unit 140 and classifies the motor imagery intention of the EEG signals using the received values. That is, by the application of the analysis model, the motor imagery intention is classified using the input of the values of the spatial features changing for each unit time.

For example, the user's intention is classified by the deep learning artificial neural network using the feature values extracted by the spatial domain feature extraction model and the temporal domain feature extraction model.

In this instance, the intention classification unit 150 classifies changes in imagined movements of only one body part as motor imagery and removes background noise generated by movements or imagination of the other body parts.

One body part refers to a single body part such as any one of the two hands, any one wrist or any one eye, and the present disclosure predicts one body part motor imagery more difficult to classify than distinguishing between many parts.

When the motor imagery intention is classified, as an embodiment, an intention execution command is transmitted to the external device such as a robotic arm (S150A), to fulfill the purpose according to the user's motor imagery.

As another preferred embodiment, the present disclosure further includes a motor imagery determination step (S110A). The motor imagery determination step (S110A) is preferably performed before feature mapping after collecting the EEG signals.

In the motor imagery determination step (S110A), the feature point generation reading unit 160 determines if the EEG signals are generated by motor imagery by analyzing signal changes in a specific frequency band included in the EEG signals, and the following feature point analysis starts.

Specifically, the frequency analysis unit 161 analyzes the frequency of the EEG signals measured by the EEG measurement device, and the energy analysis unit 162 analyzes the measured energy in α band region and β band region of the analyzed frequency. The energy analysis tracks energy increases or decreases in a band region and β band region, and α band region includes μ band region.

Accordingly, the motor imagery determination unit 163 determines if the EEG signals are generated by motor imagery by analyzing the energy changes in α band region and β band region based on the analysis result of the energy analysis unit 162.

That is, during imagination of movements, the motor cortex of the brain has energy decreases in α band region, and energy increases in β band region, and through this, the motor imagery state is determined.

According to the determination result, the computer processing throughput may be optimized by applying the motor imagery intention classification model, or a unit reference time applied to the model may be determined using data at the exact point in time.

In particular, the feature mapping unit receives time information in which the EEG signals are generated by motor imagery from the feature point generation reading unit 160, and includes only the EEG signals generated during motor imagery in a matrix structure.

That is, it is possible to precisely analyze the user's motor imagery intention by extracting and analyzing only the feature points by motor imagery and preventing noise generated at the other points in time from being included (mixed) in analysis.

The particular embodiments of the present disclosure have been hereinabove described. However, the spirit and scope of the present disclosure is not limited to these particular embodiments, and those skilled in the art will understand that various modifications and changes may be made thereto without departing from the essence of the present disclosure.

Accordingly, the disclosed embodiments are provided to help those skilled in the art to understand the scope of the present disclosure fully and completely and it should be understood that the present disclosure is provided for illustration in all aspects but not intended to be limiting, and the present disclosure will be defined by the scope of the appended claims.

### DETAILED DESCRIPTION OF MAIN ELEMENTS

110: Information storage unit
120: Feature mapping unit
130: Spatial feature analysis unit
140: Temporal feature analysis unit
150: Intention classification unit
161: Frequency analysis unit
162: Energy analysis unit
163: Motor imagery determination unit
BL: Signal block
FE: Feature point

1. A motor imagery classification apparatus using electroencephalography (EEG) for predicting a user's intention by analyzing EEG signals generated during motor imagery in chronological order, comprising:
an information storage unit (110) to collect EEG signals measured by an EEG measurement device and store the EEG signals;
a feature mapping unit (120) to classify the EEG signals into signals measured for each set unit time, combine features measured at a same unit time among features of the EEG signals arranged in chronological order and arrange the features in a matrix structure;
a spatial feature analysis unit (130) to set a matrix including the features as each layer to analyze spatial features for each layer;
a temporal feature analysis unit (140) to analyze changes in the spatial features between the layers arranged in chronological order;
an intention classification unit (150) to classify motor imagery of the measured EEG signals based on input of values of the spatial features changing for each unit time; and
a feature point generation reading unit (160) to determine if the EEG signals are generated by motor imagery by analyzing signal changes in a specific frequency band included in the EEG signals.

2. The motor imagery classification apparatus using EEG according to claim 1, wherein the feature mapping unit (120) receives time information in which the EEG signals are generated by the motor imagery from the feature point generation reading unit (160) and includes only the EEG signals generated during the motor imagery in the features arranged in the matrix structure.

3. The motor imagery classification apparatus using EEG according to claim 2, wherein the feature point generation reading unit (160) further includes:
a frequency analysis unit (161) to analyze a frequency of the EEG signals measured by the EEG measurement device;
an energy analysis unit (162) to analyze measured energy in α band region and β band region of the analyzed frequency; and
a motor imagery determination unit (163) to analyze energy changes in the α band region and the β band region, and determine that the EEG signals are generated by the motor imagery when the energy in the α band region decreases (event related desynchronization), and the energy in the β band region increases (event related synchronization).

4. The motor imagery classification apparatus using EEG according to claim 1, wherein the feature mapping unit (120) assumes that one attempt of the user to imaging moving is made for n sec, classifies the EEG signals into 2n signal blocks, and compares two successive signal blocks.

5. The motor imagery classification apparatus using EEG according to claim 1, wherein the feature mapping unit (120) applies a Common Spatial Pattern (CSP) algorithm to extract feature points of multi-channel motor imagery EEG signals.

6. The motor imagery classification apparatus using EEG according to claim 5, wherein the feature mapping unit (120) determines a weight of a CSP filter applied to the CSP algorithm, and the weight of the CSP filter is determined by a frequency of the EEG signals generated during the motor imagery.

7. The motor imagery classification apparatus using EEG according to claim 1, wherein the spatial feature analysis unit (130) extracts the spatial features for each layer by applying a Convolutional Neural Network (CNN) model to each layer.

8. The motor imagery classification apparatus using EEG according to claim 7, wherein the temporal feature analysis unit (140) extracts the temporal features using the changes in the spatial features between temporally successive layers by applying a Recurrent Neural Network (RNN) model to each layer.

9. The motor imagery classification apparatus using EEG according to claim 1, wherein the intention classification unit (150) performs classification by a deep learning artificial neural network.

10. The motor imagery classification apparatus using EEG according to claim 1, wherein the intention classification unit (150) classifies changes in imagined movements of only one body part as the motor imagery and removes background noise generated by movements or imagination of other body part.

11. A motor imagery classification method using electroencephalography (EEG) for predicting a user's intention by analyzing EEG signals generated during motor imagery in chronological order, comprising:
a motor imagery determination step (S110a) of determining, by a feature point generation reading unit (160), if the EEG signals are generated by motor imagery by analyzing signal changes in a specific frequency band included in the EEG signals;
an information storage step (S110) of collecting the EEG signals measured by an EEG measurement device and storing the EEG signals in an information storage unit (110);
a block mapping step (S120) of classifying, by a feature mapping unit (120), the EEG signals into signals measured for each set unit time, combining features measured at a same unit time among features of the EEG signals arranged in chronological order and arranging the features in a matrix structure;
a spatial domain analysis step (S130) of setting, by a spatial feature analysis unit (130), a matrix including the features as each layer to analyze spatial features for each layer;
a temporal domain analysis step (S140) of analyzing, by a temporal feature analysis unit (140), changes in the spatial features between the layers arranged in chronological order; and
a user's intention classification step (S150) of classifying, by an intention classification unit (150), the motor imagery of the measured EEG signals based on input of values of the spatial features changing for each unit time.
